(19) United States
(12) Patent Application Publication
(10) Pub. No.: US 2010/0075991 A1
(43) Pub. Date: Mar. 25, 2010

(54) Post-translational regulation of catalytic activities of cytochrome P450 46A1 and uses thereof

(76) Inventors: Irina A. Pikuleva, Lyndhurst, OH (US); Natalia V. Mast, Cleveland, OH (US)

Correspondence Address: Benjamin Aaron Adler;ADLER & ASSOCIATES, 8011 Candle Lane, Houston, TX, 77071, US

(21) Appl. No.: 12/592,325

(22) Filed: Nov. 23, 2009

Related U.S. Application Data
(63) Continuation-in-part of application No. PCT/US08/06537, filed on May 22, 2008.
(60) Provisional application No. 60/931,241, filed on May 22, 2007.

(51) Int. Cl. A61K 31/506; C12N 9/99; G01N 33/53; A61K 31/4164; A61K 31/426; C07D 403/04; C07D 277/38; C07D 233/90

(52) U.S. Cl. 514/256; 435/184; 435/7.1; 514/400; 514/370; 514/622; 544/333; 548/193; 548/336.5

(57) ABSTRACT

Provided herein are methods and compounds for post-translational regulation of cytochrome P450 46A1 (CYP46A1) enzyme activity in the brain and retina. Also, a method for identifying a potential regulator of a CYP46A1 enzyme using crystal structures of the enzyme and a subsequent method for screening for a regulatory activity in the presence of CYP46A1 enzyme are provided. In addition, the regulator compounds that either inhibit or stimulate cholesterol hydroxylation by the CYP46A1 enzyme are provided. Further provided is a method of treating a pathoneurological condition associated with increased cholesterol levels in the brain and retina using the stimulatory compounds.

### CROSS-REFERENCE TO RELATED APPLICATIONS

This continuation-in-part application claims benefit of priority under 35 U.S.C. §120 of international application PCT/US2008/006537, filed May 22, 2008, which claims benefit of priority under 35 U.S.C. §119(e) of provisional application U.S. Ser. No. 60/931,241, filed May 22, 2007, now abandoned, the entirety of both of which are hereby incorporated by reference.

### FEDERAL FUNDING LEGEND

This invention was produced in part using funds obtained through grants GM62882 and AG024336 from the National Institutes of Health. Consequently, the federal government has certain rights in this invention.

### BACKGROUND OF THE INVENTION

1. Field of the Invention

The present invention relates to the field of lipid metabolism and neurological disorders. Specifically, the present invention provides methods of post-translationally altering the activity level of cytochrome P450 46A1 enzyme (CYP46A1) and methods of treating a neurological disease or disorder resulting from such alteration.

2. Description of the Related Art

Cytochrome P450 46A1 (CYP46A1) is a membrane-associated enzyme that catalyzes cholesterol 24S-hydroxylation, the first step in the major pathway of cholesterol elimination from the brain (1). Cerebral cholesterol is turned over at a very slow rate, therefore only 5-7 mg of cholesterol is converted daily to 24S-hydroxycholesterol by CYP46A1 (2). Unlike cholesterol, 24S-hydroxycholesterol can cross the blood-brain barrier and be transported to the liver for degradation to bile acids or conjugation with the sulfate and/or glucuronic acid (3).

Medical significance of CYP46A1 in humans is not yet clear because individuals lacking the enzyme activity have not been identified. CYP46A1 gene knockout mice show severe deficiencies in spatial, associative, and motor learning (4). It is demonstrated that blocking of cholesterol breakdown in the brain reduces the synthesis of geranylgeraniol, an intermediate in the cholesterol biosynthesis pathway that is required for learning in mice and humans. A number of frequent intronic polymorphisms have been identified in CYP46A1. However, investigation of one polymorphism yielded conflicting data about a link between the polymorphism and Alzheimer's disease with about twice as many investigators postulating a link (5-20). Surprisingly, the effects of this intronic polymorphism on CYP46A1 mRNA and protein levels have not been determined.

CYP46A1 is unusual among cholesterol-metabolizing P450s. First, it is expressed almost exclusively in neural tissues, the brain and retina. Second, in healthy people, CYP46A1 immunoreactivity is predominantly confined to neurons, whereas in patients with Alzheimer's disease CYP46A1 expression is also detected in astrocytes (21-22). Third, CYP46A1 appears to have a very broad substrate specificity (23). In addition, although cholesterol is the only known physiological substrate for CYP46A1 at present, purified recombinant CYP46A1 can metabolize a number of structurally diverse drugs. Fourth, CYP46A1 activity in vitro can be reconstituted with either oxidoreductase, the redox partner for microsomal P450s, or with ferredoxin reductase and ferredoxin, the mitochondrial P450 electron transfer chain. Thus, it is possible that in vivo CYP46A1 may have a dual subcellular distribution residing in both, the endoplasmic reticulum and inner mitochondrial membrane.

Finally, regulation of CYP46A1 activity is very different from that of other family members, specifically, CYP7A1 and CYP27A1. CYP46A1 is not subject to regulation by cholesterol, oxysterols, bile acids and a wide variety of other compounds known to influence cellular cholesterol homeostasis, e.g., steroid hormones, insulin, growth hormone, thyroid hormone, and cAMP (24). Oxidative stress is the only identified factor causing significant up-regulation of CYP46A1 transcription. The low level of transcriptional control may be a consequence of the effective blood-brain and blood-retina barriers that prevent extracerebral cholesterol from fluxing into the brain. Cholesterol availability is hypothesized to be the most critical factor for production of 24S-hydroxycholesterol (24). Protein expression of CYP46A1 and plasma levels of 24S-hydroxycholestrol are highly stable in adults.

CYP46A1 activity may not be limited to cholesterol degradation. 24S-hydroxycholesterol is a potent activator of the LXR receptors (25); therefore, CYP46A1 may play a regulatory role by producing a biologically active product. It is possible that CYP46A1 may also be involved in subsequent metabolism of 24S-hydroxycholesterol because, in vitro, it converts 24S-hydroxycholesterol to 24,25- and 24,27-dihydroxycholesterols where 24S-hydroxycholesterol is a much better substrate for CYP46A1 than cholesterol. Furthermore, in vitro studies indicate that CYP46A1 has a broad substrate specificity and metabolizes a number of structurally diverse compounds including different cholesterol derivatives and drugs (23). CYP46A1 may participate in metabolism of neurosteroids and drugs that are targeted to the central nervous system.

Thus, there is a recognized need in the art to discover compounds that can bind within the CYP46A1 active site and regulate enzyme activity thereby. More specifically, the prior art is deficient in methods for post-translationally regulating CYP46A1 activity and methods of treating or preventing a pathoneurological condition resulting therefrom. The present invention fulfills this long-standing need and desire in the art.

### SUMMARY OF THE INVENTION

The present invention is directed to a method for controlling an activity of a cytochrome P450 46A1 (CYP46A1) enzyme. The method comprises contacting the CYP46A1 enzyme with a compound that binds within the CYP46A1 active site such that cholesterol hydroxylation is effectively inhibited or stimulated thereby controlling the CYP46A1 activity.

The present invention also is directed to a method of designing a potential regulator compound of a post-translational CYP46A1 activity. The method comprises identifying a test Compound that interacts within the active site of CYP46A1, said identification based at least in part on part on the crystal structure of CYP46A1 described herein. The present invention is directed to a related method comprising a further step of screening the test compounds for regulation of a post translational activity of CYP46A1 enzyme.

The present invention is directed further to a related method for screening for a compound regulating post-translational CYP46A1 activity. The method comprises selecting a designed test compound that interacts with the active site of CYP46A1 enzyme and contacting the CYP46A1 enzyme with the test compound and cholesterol or with cholesterol alone. The level of cholesterol hydroxylation is measured in the presence and in the absence of the test compound. The level of cholesterol hydroxylation in the cell in the presence of the test compound is compared with the level of cholesterol hydroxylation in the absence of the test compound. A decrease in cholesterol hydroxylation in the presence of the test compound is indicative that the test compound is an inhibitor of CYP46A1 activity. An increase in cholesterol hydroxylation in the presence of the test compound is indicative that the test compound is a stimulator of CYP46A1 activity. The inhibitor or stimulator compounds thus regulate post-translational CYP46A1 activity. The present invention is directed further to the inhibitor and stimulator compounds of CYP46A1 post-translational activity designed and identified by the screening method described herein.

The present invention is directed further still to a method for treating a pathoneurological condition associated with increased cholesterol levels in the brain or retina of a subject. The method comprises administering to the subject a pharmacologically effective amount of the screened stimulator compound described herein. The stimulator compound increases hydroxylation of cholesterol by the CYP46A1 enzyme in the brain thereby decreasing cholesterol levels therein to treat the pathoneurological condition in the subject.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIGS. 1A-1B depict the CYP46A1 active site. FIG. 1A is an enlarged view of the active site around the sulfate anion of CH-3S, and in the vicinity of the heme iron (FIG. 1B). Dashed white lines connect the C24 and C25 of CH-3S and the heme iron.

FIGS. 2A-2B are comparisons of the CH-3S-bound and ligand-free CYP46A1 structures. In FIG. 2A the superposition of the two structures. The CH-3S-bound structure is colored in cyan, heme is in pink, and CH-3S is yellow except for the sulfate group, which is in orange. The ligand-free structure is colored in grey, and heme is in light pink. FIG. 2B the solvent accessible surface of the ligand-free (in grey) and CH-3S-bound (in yellow) active sites. The volume does not change significantly from 309 A3 in the ligand-free structure to 320 A3 in the CH-3S-bound structure as calculated by VOIDOO (27). The active site residues are colored in grey in the ligand-free structure and in cyan is CH-3S-bound. Side chains in contact with the steroid nucleus shift 0.6-4.2 Å upon substrate binding, whereas residues interacting with the sulfate group shift up to 9-12 Å in the two structures.

FIG. 3 depicts the subpocket (highlighted in magenta) in the active site of CH-3S-bound CYP46A1. The surface of the active site is shown in grey mesh. The CH-3S-bound structure is colored from blue at the N-terminus to red at the C-terminus, heme is in pink, and CH-3S is yellow except for the sulfate group, which is in orange. The flexible B′-C loop is in medium blue behind the cavity.

FIGS. 4A-4E demonstrate the inhibitory effects on cholesterol hydroxylation by CYP46A1 of various sulfate-containing containing steroids (FIG. 4A), histamine receptor antagonists including those containing the azole and sulfonamide moieties, (FIG. 4B), antifungal drugs containing an azole moiety (FIG. 4C) and some other commonly used drugs including monoamine oxidase inhibitors and the antibacterial agent sulfanilamide (FIGS. 4D-4E). *indicates a drug known to be metabolized by CYP46A1 in vitro.

FIG. 5 shows the inhibition of CYP46A1 activity by voriconazole in vitro. IC50 plots for the inhibition of cholesterol 24-hydroxylation and of 25- and 27-hydroxylations of 24S-hydroxycholesterol are shown in closed squares and open circles, respectively. Voriconazole concentration is plotted on a log scale.

FIGS. 6A-6D shows the effect of 5 daily intraperitoneal injections (60 mg/kg body weight) with voriconazole. FIG. 6A: on levels of 24S-hydroxycholesterol, FIG. 6B: cholesterol, FIG. 6C: 27-hydroxycholesterol, and FIG. 6D: lathosterol in the mouse brain.

FIGS. 7A-7C show relative levels of mRNA expression in mouse brain after 5 daily intraperitoneal injections (60 mg/kg body weight) with voriconazole. FIG. 7A shows the effect on CYP46A1; FIG. 7B shows the effect on HMG CoA Reductase; FIG. 7C shows the effect on HMG CoA Synthase.

FIG. 8 shows the kinetics of brain levels of voriconazole after a single intraperitoneal injection (60 mg/kg body weight). The data is from one mouse at each time point.

### DETAILED DESCRIPTION OF THE INVENTION

As used herein, the term “a” or “an”, when used in conjunction with the term “comprising” in the claims and/or the specification, may refer to “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

As used herein, the term “or” in the claims refers to “and/or” unless explicitly indicated to refer to alternatives only or the alternatives are mutually exclusive, although the disclosure supports a definition that refers to only alternatives and “and/or.”

As used herein, the term “antagonist” refers to a biological or chemical agent that acts within the body to reduce the physiological activity of another chemical or biological substance. In the present invention, for example, an antagonist, particularly a histamine receptor antagonist, blocks, inhibits, reduces and/or decreases the activity of the cytochrome P450 46A1 (CYP46A1) enzyme in a cell containing the same. In the present invention, the antagonist combines, binds, associates with the CYP46A1 enzyme in the cell, such that the CYP46A1 is deactivated, meaning having reduced biological activity with respect to the biological activity in the cell in the absence of the antagonist. The antagonist combines, binds and/or associates with the substrate binding site within the active site of the enzyme. The terms antagonist or inhibitor can be used interchangeably herein.

As used herein, the term “contacting” refers to any suitable method of bringing one or more of the compounds described herein or other inhibitory or stimulatory agent into contact with a CYP46A1 enzyme, as described, or a cell comprising the same. In vitro or ex vivo this is achieved by exposing the CYP46A1 enzyme to the compound or inhibitory or stimulatory agent in a suitable medium. For in vivo applications, any known method of administration is suitable as described herein.

As used herein, the terms “effective amount” or “pharmacologically effective amount” are interchangeable and refer to an amount that results in an a delay or prevention of onset of the disease, disorder or condition or results in an improvement or remediation of the symptoms of the disease, disorder or condition. Those of skill in the art understand that the effective amount may improve the patient's or subject's condition, but may not be a complete cure of the disease, disorder and/or condition.

As used herein, the terms “inhibit” or “inhibitory” refers to the ability of the compound to block, partially block, interfere, decrease, reduce or deactivate cytochrome P450 46A1 (CYP46A1). Thus, one of skill in the art understands that the term inhibit encompasses a complete and/or partial loss of activity of CYP46A1. CYP46A1 activity may be inhibited by occlusion or closure of the active site, by disruption of the interaction with the substrate, by sequestering CYP46A1 and/or the substrate, or by other means. For example, a complete and/or partial loss of activity of the CYP46A1 may be indicated by a reduction in cholesterol hydroxylation. As such it will be readily apparent to one of skill in the art that the terms “stimulate”, “stimulatory” or “activate” refer to the ability of the compound to increase the activity of CYP46A1 over that occurring in the absence of the stimulatory compound. As used herein, the term “subject” refers to any target of the treatment.

In one embodiment of the present invention there is provided a method for controlling an activity of a cytochrome P450 46A1 (CYP46A1) enzyme, comprising the step of contacting the CYP46A1 ezyme with a compound that binds within the CYP46A1 active site such that cholesterol hydroxylation is effectively inhibited or stimulated thereby controlling the CYP46A1 activity.

In one aspect of this embodiment the compound may be a CYP46A1 enzyme inhibitor binding within a substrate-binding site of the enzyme active site. Also, the inhibitor may be a compound comprising one or more of a sulfate moiety, a sulfonamide moiety or an azole moiety, a histamine receptor antagonist, a monoamine oxidase inhibitor, or other drug-like compound. Examples of the sulfate-containing compound are cholesterol sulfate, pregnenolone sulfate, estradiol sulfate, testosterone sulfate, or DHEA sulfate. Examples of the sulfonamide-containing compound are famotidine or sulfanilamide. In particular an azole-containing compound may be an antifungal compound. Representative examples of the antifungal compounds are voriconazole or clotrimazole. Representative examples of the histamine receptor (R) antagonist are famotidine, nizatidine, cimetidine, ranitidine, thioperamide, or clobenpropit. Representative examples of the monoamine oxidase inhibitor are selegiline and tranylcypromide. Representative examples of the other drug-like compound are retinol or aspirin.

In another aspect of this embodiment the compound may be a CYP46A1 enzyme stimulator binding within a subpocket of the enzyme active site without interfering with substrate binding therein. In this aspect, the subpocket may be formed by at least residues L112, F121, V126, L219, I222, I301, A302, A474, and T475. Also, the stimulator may be a non-steroidal compound. Representative examples of a non-steroidal compound are acetaminophen or phenacetin.

In another embodiment of the present invention there is provided a method for designing a potential regulator compound of a post-translational CYP46A1 activity, comprising identifying a test compound that interacts within the active site of CYP46A1, the identification based at least in part on the crystal structure of CYP46A1.

Further to this embodiment the method comprises screening the test compounds for regulation of a post translational activity of CYP46A1 enzyme. In this further embodiment screening may comprise selecting a designed test compound that interacts with the active site of CYP46A1 enzyme; contacting the CYP46A1 enzyme with the test compound and cholesterol or with cholesterol alone; measuring the level of cholesterol hydroxylation in the presence and in the absence of the test compound; and comparing the level of cholesterol hydroxylation in the presence of the test compound with the level of cholesterol hydroxylation in the absence of the test compound, where a decrease in cholesterol hydroxylation in the presence of the test compound is indicative that the test compound is an inhibitor of CYP46A1 activity or where an increase in cholesterol hydroxylation in the presence of the test compound is indicative that the test compound is a stimulator of CYP46A1 activity, said inhibitor or stimulator compound thereby regulating post-translational CYP46A1 activity post-translationally. In both embodiments the CYP46A1 inhibitor or stimulator compound may cross the blood brain barrier or the blood retina barrier.

In one aspect of both embodiments the crystal structure may be 2Q9G and the test compound is an inhibitor of cholesterol hydroxylation by CYP46A1 where the inhibitor binds within a substrate binding site in 2Q9G. Also, in this aspect the inhibitor may be a substrate for CYP46A1. In another aspect the crystal structure may be 2Q9F and the test compound is a stimulator of cholesterol hydroxylation by CYP46A1, where the stimulator binds within a subpocket of the CYP46A1 active site in 2Q9G. In this aspect the subpocket may be formed by at least residues Ile301, Val215, Ile219, Ile222, Als 474, Leu112, Leu120, and Phe121.

In a related embodiment the present invention provides a regulator compound affecting the CYP46A1 activity post-translationally identified by the screening method described supra. In one aspect of this embodiment the regulator compound may be an inhibitor comprising one or more of a sulfate moiety, a sulfonamide moiety or an azole moiety, a histamine receptor antagonist, a monoamine oxidase inhibitor, or other drug-like compound. In another aspect the regulator compound may be a stimulator of CYP46A1 post-translational activity. In this aspect the stimulator may be a non-steroidal compound having an aromatic or aryl structure.

In yet another embodiment of the present invention there is provided a method for treating a pathoneurological condition associated with increased cholesterol levels in the brain or retina of a subject, comprising administering to the subject a pharmacologically effective amount of the stimulator compound described supra, where the stimulator compound increases hydroxylation of cholesterol by the CYP46A1 enzyme in the brain or retina thereby decreasing cholesterol levels therein to treat the pathoneurological condition in the subject.

In this embodiment the increased cholesterol levels may result from the competitive binding of another drug to the substrate binding site of the CYP46A1 enzyme. Also, in this embodiment the stimulator compound may delay or prevent onset of the pathoneurological condition in the subject. Examples of the pathoneurological condition are Alzheimer's disease, dementia, deficiency in spatial, associative and motor learning, and age-related macular degeneration.

Provided herein are methods and compounds for regulating an activity level of the enzyme cytochrome P450 46A1 (CYP46A1). CYP46A1 regulation is useful in maintaining cholesterol homeostasis in the brain or retina and treating pathophysiological conditions resulting from disruption in cholersterol homeostasis. As such, two crystal structures of CYP46A1 have been deduced. The 1.9 Å-resolution structure of substrate cholesterol sulfate-bound CYP46A1 and the 2.4 Å-resolution structure of ligand-free CYP46A1 are useful in designing or otherwise identifying effective regulatory compounds. The atomic coordinates and structure factors have been deposited in the Protein Data Bank with PDB ID codes 2Q9F and 2Q9G, respectively.

Also, a method for designing compounds that regulate activity of CYP46A1 post-translationally is provided. It is contemplated that techniques known in the art may be expanded to identify additional molecules that can act as lead compounds for the development of novel CYP46A1 regulatory compounds that can be used for experimental and clinical purposes. Alternatively, known compounds with a demonstrable inhibitory or stimulatory effect on CYP46A1 activity or are more efficient metabolites of CYP46A1 may be useful as lead compounds. For example, and without being limiting, these inhibitors may be used to design other, more potent regulators based on the CYP46A1 crystal structures.

Designed or selected compounds have the potential to bind within the active site of CYP46A1. As inhibitors or additional substrates, regulatory compounds may compete with cholesterol for the substrate binding site. As stimulators, the regulatory compounds may bind within a subpocket of the cholesterol sulfate-bound CYP46A1 structure without interfering with cholesterol binding. For example, the 2.4 Å-resolution crystal structure, such as 2Q9G, of CYP46A1 is useful in designing inhibitors while the 1.9 Å-resolution crystal, structure, such as 2Q9F, of substrate (cholesterol sulfate)-bound CYP46A1 is useful in designing stimulators.

In addition, a method for screening for compounds that regulate the activity of CYP46A1 enzyme is provided. These regulatory compounds may act as inhibitors or stimulators of CYP46A1 activity or may be a substrate metabolized by the enzyme. Potential compounds may be known in the art, known or designed derivatives or analogs thereof or designed de novo using well-known and standard computer aided drug design techniques and programs based on the deduced crystal structures of CYP46A1. Potential compounds not readily commercially available may be chemically synthesized using any suitable chemical synthetic process.

The efficacy of a potential regulatory compound may be tested using standard enzyme assays well-known in the art. For example, CYP46A1-associated cholesterol hydroxylation activity may be assayed in the presence of cholesterol and in the presence or absence of a potential inhibitor. A decrease in cholesterol hydroxylation in the presence of the potential inhibitor compared to cholesterol hydroxylation in the absence of the potential inhibitor is indicative that it has an ability to inhibit CYP46A1 substrate binding within the substrate binding site of CYP46A1.

Alternatively, cholesterol hydroxylation activity by CYP46A1 may be assayed in the presence of cholesterol and in the presence or absence of a potential stimulator or test compound. An increase in cholesterol hydroxylation in the presence of the potential stimulator compared to cholesterol hydroxylation in the absence of the potential stimulator is indicative that it has an ability to stimulate CYP46A1-associated cholesterol hydroxylation. It is contemplated that binding of these drugs near cholesterol, in the active site of CYP46A1, either reduces cholesterol freedom of motion in the active site, or affects the hydration state of the active site and thus increases the affinity of cholesterol for CYP46A1 and the rate of cholesterol catalysis.

The regulatory compounds of the present invention may be inhibitors or stimulators of CYP46A1 activity or may function as endogenous substrates. For in vivo regulation these compounds must be able to cross the blood-brain (BBB) and blood-retina (BRB) barriers upon systemic administration, or be present in the brain or retina, as CYP46A1 activity occurs in the brain and in the retina. In addition compositions comprising the regulatory compounds, as provided herein, and a pharmaceutically acceptable carrier are contemplated.

For example, an inhibitory compound may be a sulfate- or sulfonamide (SO₂NH₂)-containing compound, a steroid-like compound or other aromatic or aryl compound or a derivative or an analog thereof, a histamine receptor antagonist, particularly an H₂R or H₃R antagonist, including azole-containing and non-azole-containing HR antagonist compounds, an azole-containing compound, for example, an antifungal azole-containing compound, a monoamine oxidase inhibitor or other non-steroidal aromatic or aryl compound structurally similar to known drugs or drug-like compounds.

Stimulatory compounds may be non-steroidal compounds or ligands that bind within a subpocket of the active site and stimulate cholesterol hydroxylation by CYP46A1 in the brain and retina. The sub-pocket of the enzyme active site is formed by at least residue Ile301 from the I helix and residues Val215, Ile219, Ile222 from the F helix, residue Ala474 from the loop between the β4-1 and β4-2 strands, and residues Leu112, Leu120, Phe121 from the B′-C loop. For example a stimulatory compound may be a non-steroidal aromatic or aryl compound that may be structurally similar to known drugs or drug-like compounds.

Also provided are methods of treating a pathoneurological condition such as brain degenerative diseases or disorders associated with a disruption of cholesterol homeostasis in the brain and/or retina. Without being limiting, such conditions may be associated with an increase in cholesterol levels in the brain and/or retina, for example, Alzheimer's disease, dementia, deficiency in spatial, associative and motor learning, or age-related macular degeneration. Administration of a pharmacologically effective amount of a stimulatory compound to a subject at risk for such condition or exhibiting symptoms of the same stimulates cholesterol hydroxylation.

Thus, it is contemplated that the regulatory compounds described herein are effective to reduce or prevent adverse effects upon cholesterol homeostasis occurring upon the binding or competitive binding of those drugs that are able to cross the blood brain or blood retina barriers to the CYP46A1 enzyme in the neural tissues. Although providing a therapeutic benefit elsewhere in a subject, the concomitant binding to CYP46A1 in the subject increases the risk of developing the pathoneurological conditions described herein. Therefore, it is particularly contemplated that those stimulatory compounds effective to increase cholesterol hydroxylation would exhibit an ameliorative effect in the presence of these other CYP46A1 substrates.

The method of the present invention employs the compounds identified herein for both in vitro and in vivo applications. For in vivo applications, the invention compounds can be incorporated into a pharmaceutically acceptable formulation for administration. Those of skill in the art can readily determine suitable dosage levels when the invention compounds are so used. As employed herein, the phrase “suitable dosage levels” refers to levels of compound able to cross the blood brain or blood retina barriers that are sufficient to provide circulating concentrations high enough to effectively stimulate CYP46A1 activity in vivo. Also, formulations and delivery vehicles for the regulatory compounds provided herein useful for in vivo applications may be any that are suitable for the application and well-known and standard in the art.

Also, as is well known in the art, a suitable dosage level of active compounds such as a CYP46A1 stimulator or related-compounds thereof for any particular patient depends upon a variety of factors including the activity of the specific compound employed, the age, body weight, general health, sex, diet, time of administration, route of administration, rate of excretion, drug combination, and the severity of the particular disease or disorder undergoing therapy. The person responsible for administration will determine the appropriate dose for the individual subject. Moreover, for human administration, preparations should meet sterility, pyrogenicity, general safety and purity standards as required by FDA Office of Biologics standards.

The following examples are given for the purpose of illustrating various embodiments of the invention and are not meant to limit the present invention in any fashion.

### Example 1

### Methods

### Protein Purification and Crystallization

CYP46A1 complexed with CH-3S was expressed, purified and crystallized. The substrate-free form was purified using the same protocol as CH-3S-bound form except that the substrate was omitted from all the buffers, and 30 mM histidine was used to elute the enzyme from the Ni-agarose column. Crystals of substrate-free CYP46A1 were obtained under similar conditions to those of the CH-3S-bound CYP46A1, by microseeding with a cat whisker. The well solution was 8% PEG 8,000, 50 mM potassium phosphate buffer (KPᵢ), pH 4.7, 20% glycerol.

### Spectral Binding Studies

Binding affinities of different compounds were estimated as described (23, 29) using 0.25 μM P450. Titrations of CYP46A1dH were carried out in 50 mM KPᵢ pH 7.2, containing 100 mM NaCl and 0.02% Cymal-6. Steroids were added from 0.2-5 mM stocks in 2.5-45% aqueous 2-hydroxypropyl-β-cyclodextrin; clobenpropit, thioperamide, phenacetin, acetaminophen, 4′-(2-hydroxyethoxy)-acetanilide, quinine, quinidine, lansoprazole, and dapsone were dissolved in water, and tranylcypromine in 50% MetOH. The K_{d} and maximal absorbance change were estimated by non-linear least squares fitting using the quadratic form of the single-site binding equation (30).

### Kinetic Studies

The ability of recombinant CYP46A1 to metabolize CH-3S was tested using the reconstituted with cytochrome P450 reductase in vitro system. After termination of the enzyme reaction, the substrate and products were extracted, solvolyzed, converted into trimethylsilyl ethers and analyzed by gas chromatography-mass spectrometry as described (23). Kinetic parameters for cholesterol, 24OH—CH, and CH-3S were determined at 37° C. in detergent-free 50 mM KPᵢ containing 50 mM NaCl, if Δ(2-50)CYP46A1 dH was used, or in the presence of 40 μg dilauroylglycerol-3-phosphatidylcholine, 100 mM NaCl and 0.02% Cymal-6, if full-length CYP46A1 was used. The reaction conditions were optimized for the formation of only one product.

The reconstituted system (1 ml) contained 0.1-0.25 μM P450, 0.5 μM NADPH cytochrome P450 oxidoreductase, varying concentrations of cold substrate (1-75 μM), 250,000 cpm of radiolabeled substrate, and 2U of catalase. The enzymatic reaction was initiated by 1 mM NADPH, carried out for 5-15 min and terminated by vortexing with 2 ml of CH₂Cl₂, if cholesterol or 24OH—CH was used, or with butanol containing 0.3 M NaCl, if CH-3S was used. The organic phase was isolated, evaporated, dissolved in acetonitrile, and subjected to HPLC. Incubations with cholesterol and 24OH—CH were separated as described (38), and those with CH-3S using a linear gradient between solvent A (CH₃OH:CH₃CN:H₂O, 40:10:50, V/V) and solvent B (100% CH₃OH) over 15 min, after which the flow was kept at 100% solvent B for another 7 min. Substrate metabolism was <18% and linear with reaction time and enzyme concentration. Data were analyzed as described (23).

### Inhibition/Stimulation Studies

Effect of different compounds on cholesterol hydroxylase activity of CYP46A1 was evaluated in the reconstituted system comprising 0.25 μM full-length recombinant CYP46A1, 0.5 μM NADPH cytochrome P450 oxidoreductase, 2.7 μM cholesterol as a substrate, trace amounts of [³H]cholesterol (250,000 cpm), and 43 μM test compound. The assay buffer was the same as in kinetic studies.

### Example 2

### Design, Characterization and Crystallization of a Modified CYP46A1 Crystallography of Δ(2-50)CYP46A1Dh

Crystallographic studies were carried out on Δ(2-50)CYP46A1Dh, a modified recombinant human CYP46A1, in which the first 50 N-terminal amino acid residues were deleted, and a 4× His-tag was added at the C-terminus. The truncation removed a 23-residue transmembrane anchoring domain and rendered this membrane P450 more soluble. These modifications did not adversely affect the kinetic properties of cholesterol, 24OH—CH and CH-3S hydroxylation as shown in Table 1.

TABLE 1

| | Cholesterol | | | 24OH—CH | | | CH—3S | | |
|---|---|---|---|---|---|---|---|---|---|
| | k_{cat}, | Kₘ, | k_{cat}/Kₘ, | k_{cat}, | Kₘ, | k_{cat}/Kₘ, | k_{cat}, | Kₘ, | k_{cat}/Kₘ, |
| CYP46A1 | min⁻¹ | μM | min⁻¹/μM | min⁻¹ | μM | min⁻¹/μM | min⁻¹ | μM | min⁻¹/μM |
| Full-length¹ | 0.11² | 5.4 | 0.02 | 0.92 | 3.9 | 0.24 | 0.46 | 4.9 | 0.09 |
| Δ(2-50)¹ | 0.43 | 7.7 | 0.06 | 0.85 | 1.5 | 0.56 | 2.5 | 3.3 | 0.8 |

¹Contains a C-terminal 4x His-tag, which does not affect the kinetics of hydroxylation.
²The results are means of 3-4 measurements.
SD ≦ 20%.

Δ(2-50)CYP46A1dH was purified and crystallized in the presence of CH-3S, which binds tightly to the enzyme with an estimated K_{d} of 7 nM, which is ±10 times lower than the K_{d} of the endogenous substrate cholesterol (100 nM). CH-3S is metabolized by both full-length and truncated CYP46A1 in the reconstituted with cytochrome P450 reductase in vitro system. The catalytic efficiency of CH-3S hydroxylation by full-length and Δ(2-50)CYP46A1dH was better than that of cholesterol hydroxylation and comparable to the efficiency of 24OH—CH hydroxylation (Table 1).

It has been established that 24OH—CH can be further metabolized by CYP46A1 to 24,25- and 24,27-dihydroxycholesterols in both cell cultures and the in vitro reconstituted system. Similarly to cholesterol, the major product in the incubations with CH-3S had a retention time and mass spectrum consistent with hydroxylation at C24 (not shown). There was also a smaller conversion into a product with a retention time and the mass spectrum indicative of 24,25-dihydroxycholesterol suggesting sequential hydroxylation of the 24-hydroxycholesterol sulfate. About 10% of 24OH—CH present in human circulation is sulfated, and bovine brain contains a similar fraction (˜14%) of sulfated 24OH—CH. It is not, however, clear whether sulfated 24OH—CH is formed by the action of CYP46A1 on CH-3S or by the action of a sulfotransferase on 24OH—CH. Following successful crystallization of the CH-3S-CYP46A1 complex, the substrate-free enzyme also was crystallized.

### CH-3S Binding to Δ(2-50)CYP46A1dH

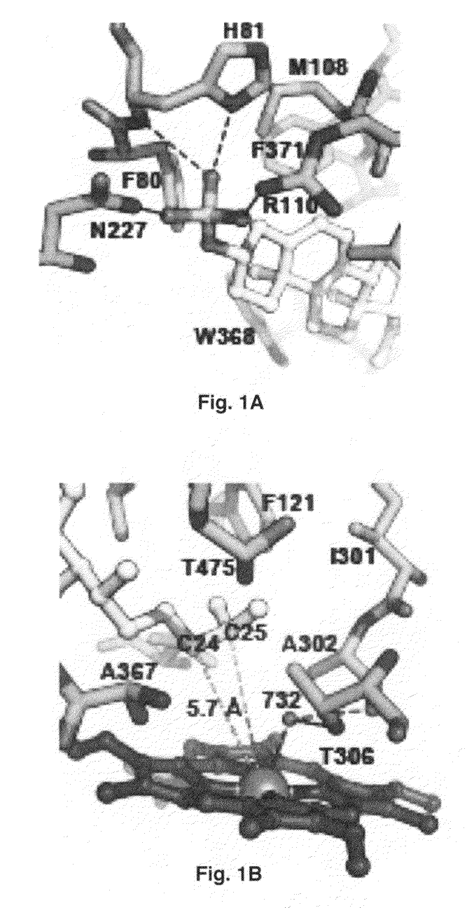

CH-3S occupies the active site cavity over its entire length with the steroid side chain facing the distal surface of the heme prosthetic group and the sulfate anion directed toward the protein surface. The sulfate group forms four hydrogen bonds, with His81 (β1-1-β1-2 loop), Arg110 (B′ helix), and Asn227 (F-G loop), of the enzyme (FIG. 1A).

The steroid nucleus interacts with Phe80 (β1-1-β1-2 loop), Met108, Tyr109, Ala111, Leu112 (B′ helix), Ile222 (F helix), Trp368, Phe371 (β1-4 strand), and Ala474 (β4 loop). Three of these residues, Ala111, Leu112, and Ile222, contact the flat surface of the steroid nucleus and three, Phe80, Trp368, Phe371, are on the opposite side contacting steroid axial methyl groups. Met108 and Tyr109 restrain the steroid nucleus along one edge as does Ala474 at the edge of the C ring. A hydrogen bond between Trp368 and Ala474, and a network of hydrogen bonds involving Tyr109, Thr370, Phe371, Arg372 and a heme propionate position these active site residues.

The aliphatic tail of CH-3S is surrounded by Phe121, Val126 (in a B′-C loop insertion, unique to CYP46A1), Ile301, Ala302, T306 (1 helix), Ala367 (β4-1 strand), and Thr475 (β4 loop) which are located at a 3.7-4 Å distance and likely to limit its motion. The C24 and C25 atoms of CH-3S, the primary and secondary sites of hydroxylation by CYP46A1, respectively, are positioned at a 5.7±0.05 Å distance from the heme iron (FIG. 1B). The orientation and position of CH-3S suggest that cholesterol will have a similar overall mode of binding. A difference could be in contacts of the cholesterol 3β hydroxyl with CYP46A1, and if so, in the depth of insertion in the active site. Residues that may be involved in recognition of the cholesterol 3β hydroxyl are His81 and Asn227.

### Ligand-Free CYP46A1 Structure in Comparison to the Substrate-Bound Form

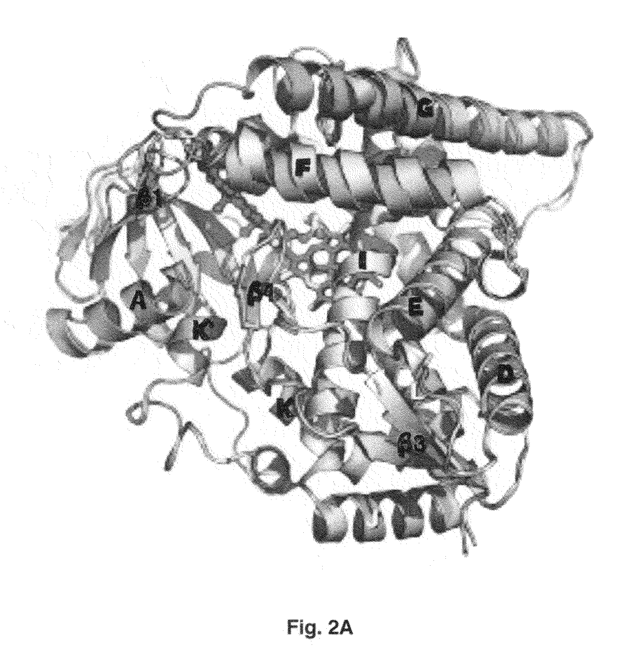

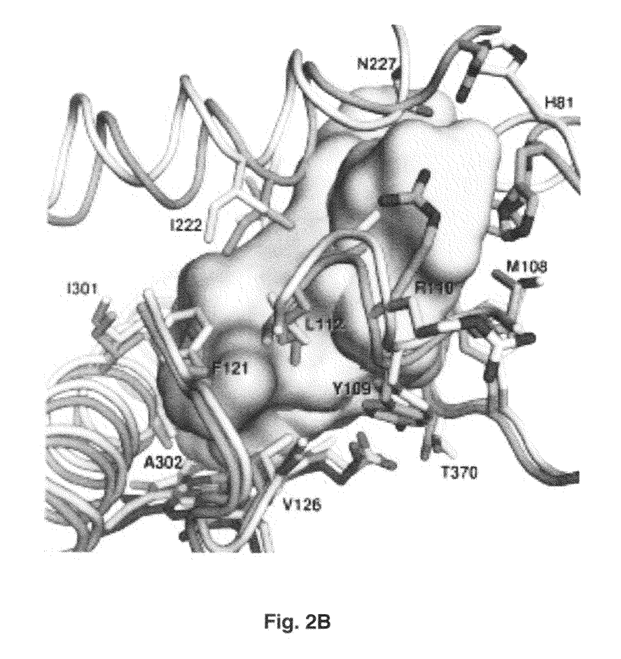

Major differences in the substrate-bound vs. unliganded structures are observed in the positions of the secondary structure elements that define the entrance to the active site cavity, helices B′ and F (residues 106-113 and 209-225, respectively), and the loop linking sheets β1-1 and β1-2 (residues 79-83) (FIG. 2A). Binding of CH-3S induces concerted movement of helix B′ and the F-G loop inward and the β1-1-β1-2 loop outward.

These movements are accompanied by shortening of the sheets β1-1 and β1-2 and elongation of the G helix by 1.5 turns, which together with the F helix, also shifts toward the β-sheet domain. The F-G loop becomes more stabilized in the CH-3S structure and could be traced; in the substrate-free structure electron density for residues 230-239 is not observed. Substrate binding results in a formation of the channel that extends ˜25 Å from the heme Fe to the protein surface. Although the shape of the active site cavity changes when CH-3S binds (FIG. 2B), the volume of the active site does not change appreciably as calculated by VOIDOO (38).

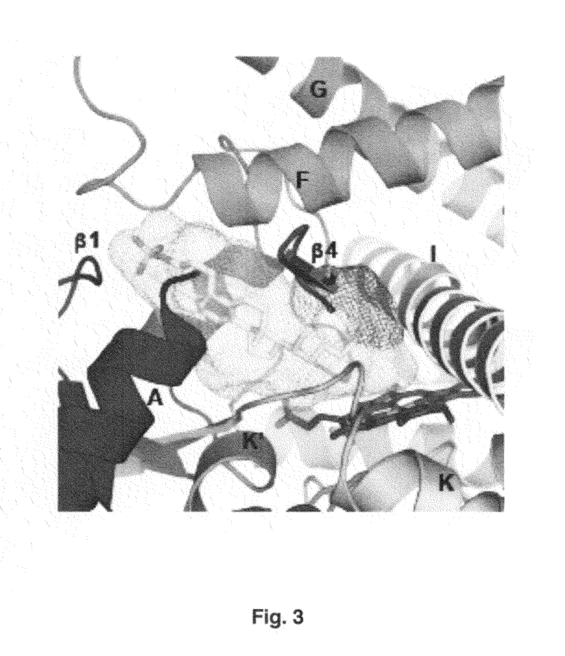

There is an unfilled space, or a subpocket, in the active site of CH-3S-bound CYP46A1 (FIG. 3). The subpocket is adjacent to the CH-3S side chain and delimited by segments of the I helix (Ile301) and F helix (Val215, Ile219, Ile222), the loop between the β4-1 and β4-2 strands (Ala474), and a part of the B′-C loop (Leu112, Leu120, Phe121).

### Example 3

### Cholesterol Hydroxylation by CYP46A1 in the Presence of Pharmaceutical and Non-Pharmaceutical Compounds

### Enzyme Assay Measuring Cholesterol Hydroxylation

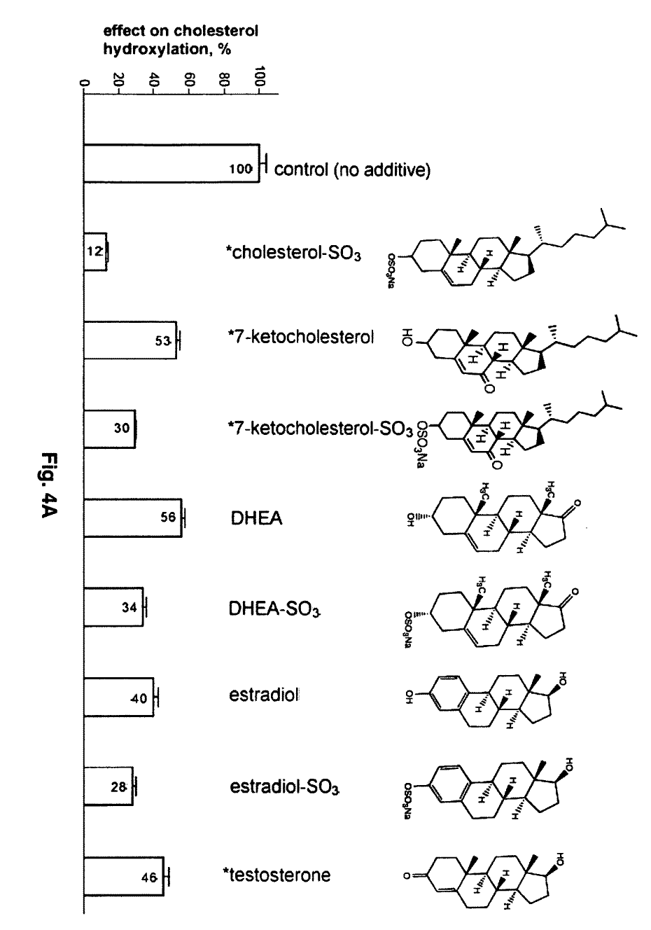

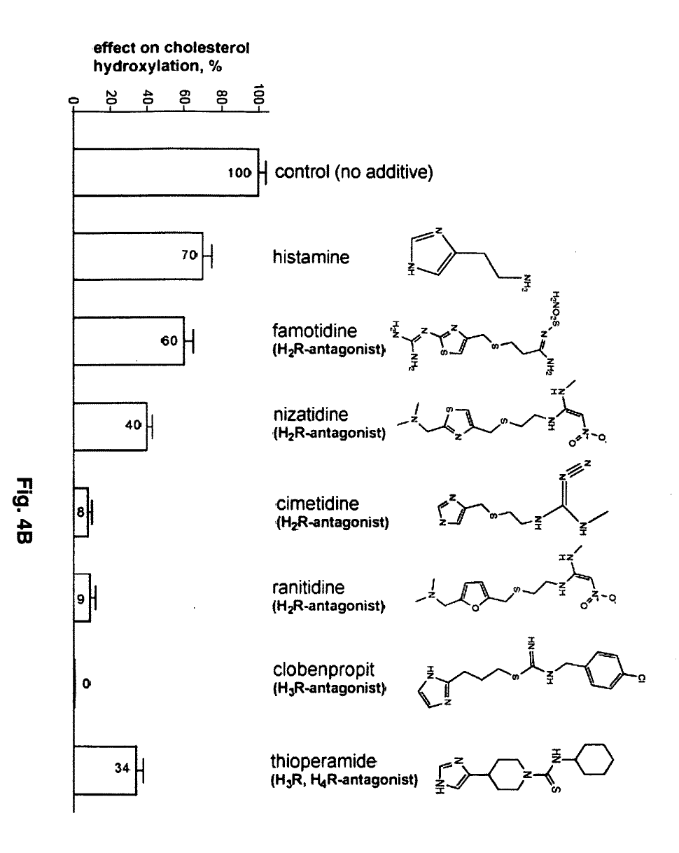

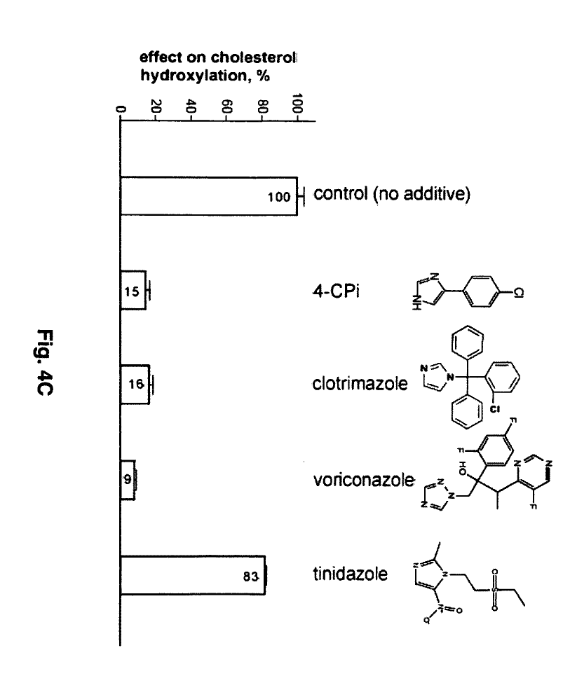

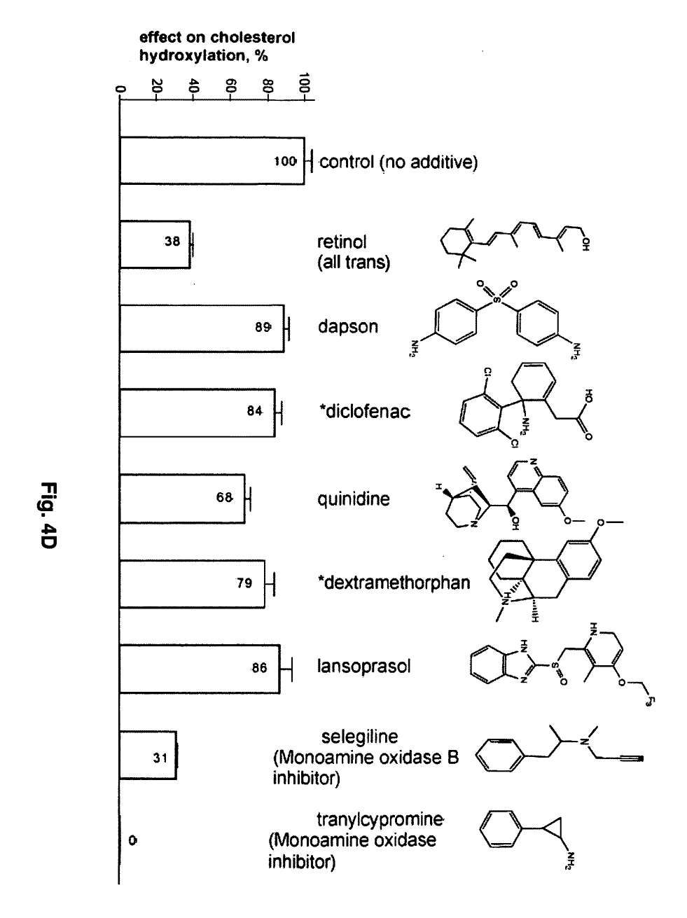

Conformational flexibility of the active site suggested a potential for the enzyme to accommodate ligands other than sterols. Thus, the inhibitory or stimulatory properties of more than 50 compounds, both marketed drugs and non-pharmaceuticals, were evaluated in an assay employing a fixed concentration of cholesterol as a substrate (2.7 μM, equal to 0.5 Kₘ), and fixed concentration of the potential inhibitor (43 μM). Table 2 (and FIG. 4A) shows the effect of different steroids on cholesterol hydroxylase activity and binding to CYP46A1.

TABLE 2

| | CHO | |
|---|---|---|
| Added steroid | hydroxylation¹, % | Spectral K_{d}² (Kᵢ³), μM |
| None | 100 ± 3 | |
| Cholesterol | 27 ± 3 | 0.67 ± 0.02 |
| Cholesterol-SO₄ | 13 ± 1 | 0.05 ± 0.003 |
| Pregnenolone | 32 ± 1 | Not determined, no spectral |
| | | response |
| Pregnenolone-SO₄ | 8 ± 2 | (2.5 ± 0.4) |
| DHEA | 58 ± 3 | Not measured |
| DHEA-SO₄ | 33 ± 3 | Not measured |
| Estradiol | 32 ± 3 | Not measured |
| Estradiol-SO₄ | 27 ± 2 | Not measured |
| Testosterone | 49 ± 4 | Not measured |
| Testosterone-SO₄ | 40 ± 2 | Not measured |

^{1,2}Conditions of the enzyme and spectral binding assays were the same as in Table 2 of the manuscript.
³Estimate of the Kᵢ was obtained based on determination of the IC₅₀ value (performed at 5.4 μM cholesterol (at Kₘ) and 15 concentrations of pregnenolone sulfate (0, 0.2-100 μM). The Kᵢ was then calculated using the following equation Kᵢ = IC₅₀/2 assuming competitive inhibition. The results are means of triplicate or quadruple experiments.

### Inhibitors of Cholesterol Hydroxylation by CYP46A1

FIGS. 4A-4E are graphical comparisons of the inhibitory effects of various compounds. FIG. 4A compares steroids and their sulfate derivatives, i.e., cholesterol and cholesterol sulfate, pregnenolone and pregnenolone-sulfate, DHEA and DHEA sulfate, estradiol and estradiol sulfate, and testosterone and testerone sulfate. All compounds inhibited cholesterol hydroxylation with the steroid sulfates demonstrating greater inhibitory effects than the corresponding steroids. The degree of inhibition and the Kd of cholesterol sulfate suggests that this steroid could be endogenous substrate for CYP46A1.

FIG. 4B compares the inhibitory effects of HR antagonists, i.e., azole-containing H₂R antagonists cimetidine, non-azole-containing H₂R antagonists famotidine, nizatidine and ranitidine, the azole-containing H₃R antagonist clobenpropit and the azole-containing H₃R, H₄R antagonist thioperamide in the enzyme assay. Histamine was also included for comparison. All compounds inhibited cholesterol hydroxylation with cimetidine, ranitidine and clobenpropit demonstrating greater than 90% inhibition of the CYP46A1. FIG. 4C compares the inhibitory effects of anti-fungal azole-containing compounds clotrimazole, voriconazole, related non-drug compounds, 4-(4-chlorophenyl)imidazole (4-CPi) and an antiparasitic azole-containing drug tinidazole. Except for tininazole, all compounds demonstrated significant inhibition of cholesterol hydroxylation. FIGS. 4D and 4E compare the inhibitory effects of different marketed drugs. All of them, except monoamine oxidase inhibitors selegiline and tranylcypromine, demonstrated only a modest, i.e., up to 40%, inhibition of the CYP46A1 activity. Information on pharmacokinetics of tranylcypromine in humans is available. The peak plasma concentrations of tranylcypromine Ile in the 0.065-0.19 μg/mL (0.49-1.43 μM) range (40) indicating that it has a potential to inhibit CYP46A1 in vivo.

### Stimulators of Cholesterol Hydroxylation by CYP46A1

It also was determined that three compounds modestly activate cholesterol hydroxylation by CYP46A1. Cholesterol 24-hydroxylation was increased by >30% in the presence of phenacetin or acetaminophen. Testing of nine non-pharmaceutical analogs of phenacetin led to identification of an additional activator. Table 3 shows the increased cholesterol hydroxylation by CYP46A1 in the presence of phenacetin and phenacetin-like compounds The phenacetin analog, 4′-(2-hydroxyethoxy)-acetanilide, caused even greater, 45%, stimulation of the cholesterol hydroxylase activity of CYP46A inhibited CYP46A1. While a larger activation would probably be required to significantly affect cholesterol turnover in vivo, this demonstrates that activation of CYP46A1 is possible in principle.

TABLE 3

| Drug or related compound | | | | | Activity, |
|---|---|---|---|---|---|
| (indication or use) | | Structures | | | % |
| Phenacetin (first non-steroidal anti- inflammatory drug) | 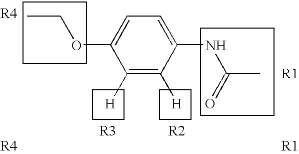 | | | | 135 ± 5 |
| Phenacetin analog 1 | HO—(CH₂)₂—O | NH—C(O)—CH₃ | H | H | 145 ± 5 |
| Acetaminophen (active | HO | NH—C(O)—CH₃ | H | H | 132 ± 4 |
| ingredient of Tylenol) | | | | | |
| Phenacetin analog 2 | CH₃ | NH—C(O)—CH₃ | H | H | 112 ± 7 |
| Phenacetin analog 3 | HO—CH₂ | NH—C(O)—CH₃ | H | H | 112 ± 4 |
| Phenacetin analog 4 | CH₃—(CH₂)₃—O | NH—C(O)—CH₃ | H | H | 107 ± 3 |
| Mexiletine (antiarrhythmic) | CH₃—CH(NH₂)—CH₂—O | H | CH₃ | CH₃ | 107 ± 3 |
| Phenacetin analog 8 | H | NH—C(O)—CH₃ | OH | H | 103 ± 2 |
| Phenacetin analog 5 | CH₃—CH₂—O | NH—C(O)—CH₂—CH₃ | H | H | 101 ± 4 |
| Phenacetin analog 9 | CH₃—O | NH—C(O)—CH₃ | H | H | 100 ± 5 |
| Phenacetin analog 7 | H | NH—C(O)—CH₃ | H | OH | 87 ± 6 |
| Phenacetin analog 6 | CH₃—CH₂—O | NH₂ | H | H | 50 ± 7 |

Without being limited by theory, the mechanism for this activation could be similar to that proposed for the stimulation of the CYP2C9-mediated 4′-hydroxylation of flurbiprofen by dapsone (41-43). The stimulation is suggested to occur via simultaneous binding of dapsone and flurbiprofen to the active site of CYP2C9 with dapsone binding limiting the motion of flurbiprofen and affecting the hydration of the active site. The subpocket in the active site could serve as a site for binding of small xenobiotics in the presence of cholesterol, where they might influence the position of the aliphatic tail of the cholesterol to improve the efficiency of hydroxylation. Although this subpocket is small, it increases in size if the substrate moves closer to the heme during activation occurring throughout optimization of the substrate position for reaction. Additionally, the novel insertion in the helix B′-C loop is likely to be sufficiently malleable to deform in the presence of the activator because it exhibits a loop structure as seen in the rearrangement upon CH-3S binding. The other possibility is that the co-activators exert their effect through some other mechanism that does not involve the subpocket.

### Voriconazole is an Efficient Inhibitor of Brain Cholesterol 24S-Hydroxylase (CYP46A1)

Voriconazole (vFEND) for in vitro studies was obtained from Pfizer. Aqueous solution of methanol (50%) was used to dissolve the compound. Voriconazole for the animal studies was obtained from the Division of Clinical Pharmacology at Karolinska University Hospital in Huddinge. The material was dissolved in a mixture of saline containing 10% ethanol and 1% serum albumin. The final concentration of voriconazole was 100 mg/ml. Recombinant full-length human CYPs 46A1 and 27A1 and bovine CYP11A1 were expressed and purified.

### Spectral Binding Assay

Binding affinities of antifungal drugs were estimated as described (5, 8) using 0.3 μM purified P450. Titrations of CYP46A1 were carried out in 50 mM potassium phosphate buffer (KPi), pH 7.2, containing 100 mM NaCl and 0.02% Cymal 6. CYPs 11A1 and 27A1 were in 50 mM KPi without any additives. Antifungal drugs were added from 0.2-5 mM stocks in 50% methanol. The apparent Kd and maximal absorbance change were estimated by non-linear least squares fitting using the quadratic form of the single-site binding equation (9).

### Determination of the In Vitro IC50 and Ki

Cholesterol hydroxylation by CYP46A1 was assayed for 20 min at 37° C. in 1 ml of 50 mM KPi, pH 7.2, containing 100 mM NaCl, 0.02% Cymal 6, 40 μg dilauroylglycerol-3-phosphatidylcholine and 2U of catalase. Purified 0.4 μM CYP46A1 was reconstituted with 0.8 μM NADPH cytochrome P450 oxidoreductase, 5.4 μM cholesterol, trace amounts of [3H]cholesterol (250,000 cpm), and varying concentrations of voriconazole (from 0.000001 μM to 100 μM). The enzymatic reaction was initiated by NADPH (final 1 mM) and terminated by vortexing with 2 ml of CH₂Cl₂. The organic phase was isolated, evaporated, dissolved in methanol, and subjected to HPLC. Under the assay conditions used, two types of products were formed, 24-hydroxycholesterol and 24, 25- and 24, 27-dihydroxycholesterols, which are produced upon further hydroxylation of 24-hydroxycholesterol. Dihydroxycholesterols were eluted as one peak during HPLC, therefore, two IC50 values were estimated, one for the inhibition of cholesterol 24-hydroxylation and the other for the inhibition of 25- and 27-hydroxylations of 24S-hydroxycholesterol. In addition, the Ki for cholesterol 24-hydroxylation was estimated assuming a model of competitive inhibition.

### Animal Experiments

Seven week old male C57/B6 J mice from Charles River were injected intraperitoneally with voriconazole (60 mg/kg or 75 mg/kg, corresponding to 0.6 and 0.75 ml, respectively, of the drug solution). The control mice were injected with the same solutions containing no voriconazole. The mice were sacrificed by cervical dislocation. Brain and liver were collected, snap frozen in liquid nitrogen and stored at −80° C.

### Lipid Extraction

Lipids were extracted from the brain according to Folch with some modifications. Approximately 10 mg of brain tissue was added to 1 ml of homogenization buffer (5 mM EDTA, 50 μg/ml butylated hydroxytoluene in phosphate-buffered saline, pH 7,4) in a clean glass tube, and the tissue was disrupted using a polytron homogeniser. Three milliliters of chloroform:methanol (2:1, v:v) were added to the homogenate and the vials were mixed by vortexing. Samples were centrifuged at 10,000 g for 10 min. The organic phase was transferred to a new vial. The aqueous phase was re-extracted as described previously three more times. The pooled organic phase was dried under stream of argon gas, and chloroform:methanol (2:1, v:v) added to the dried samples to achieve suitable concentration.

### Sterol Analysis

Sterols were analyzed by isotope dilution mass-spectrometry. Cholesterol was determined after saponification using [2H6] cholesterol as internal standard; [2H3] lathosterol was used as internal standard for quantification of lathosterol. 24S-Hydroxycholesterol and 27-hydroxycholesterol, squalene, lanosterol and dehydrolanosterol in the brain were measured by gas chromatography-mass spectrometry (GC-MS).

### Analysis of Voriconazole

Voriconazole was determined in serum and brain homogenates after the extraction with chloroform/methanol (2:1, v:v) and ethanol (10%, v/v, final), respectively.

Voriconazole extract (100 μL) was then mixed with 200 μL acetonitrile, centrifuged at 10,000 g for 10 min, and 1 μL of the supernatant injected onto a Phenomenex Luna C18 3 μm column (100×2 mm) connected to the Agilent 1100 liquid chromatography-mass spectrometry system with an autosampler and solvent degasser. Gradient elution was with initial 35% B increasing to 100% B in 5 min (A=1% acetonitrile with 25 mM formic acid; B=100% acetonitrile with 25 mM formic acid). A structure analogue (UK115794, Pfizer Ltd) was used as internal standard. Ions were monitored in a positive mode at m/z 350 (voriconazole) and m/z 348 (internal standard).

### Gene Expression Analysis

Total RNA was obtained from brain and liver tissue using Trizol (Invitrogen, Carlsbad, Calif.). cDNA was synthesised from 2 μg of total RNA using Capacity cDNA ReverseTranscription Kit (Applied Biosystems, Foster City, Calif.). Steady-state mRNA levels were estimated using Taqman probes (primer sequences available on request). All assays were run on an ABI Prism 7000 Sequense Detection System (Applied Biosystems; Foster City, Calif.). Estimation of a relative gene expression was performed using the comparative threshold cycle method, using hypoxathine-guanine phosphoribosyl transferase as endogenous control.

### Statistics

Gene expression data is expressed as mean±range (16). Sterol determinations are presented as mean±standard error of the mean (SEM). For statistical comparisons a two tailed student's T-test were preformed. All in vitro assays were performed in triplicate.

### Binding of Antifungal Azoles to Purified CYP46A1 and Other Cholesterol-Metabolizing P450s

Four antifungal azoles that are used systemically were used: voriconazole, fluconazole, ketoconazole, and itraconazole. Of them, voriconazole had the highest affinity to CYP46A1, comparable to that of clotrimazole, with the estimated Kd value of 50 nM. Voriconazole induced a type II spectral response with a minimum at 413 nm and a maximum at 434 nm. This spectral response indicates that voriconazole binds to the CYP46A1 active site and coordinates the P450 heme iron with one of its nitrogen atoms. CYP46A1 is not the only cholesterol-metabolizing enzyme expressed in the brain. Also present in the brain are CYP27A1 that catalyzes cholesterol 27-hydroxylation and CYP11A1 that converts cholesterol to pregnenolone. Similar to CYP46A1, voriconazole induced a type II spectral response in CYP11A1, but its binding was ˜100-fold weaker than that to CYP46A1 as assessed by the apparent Kd equal to 4.8 μM. In CYP27A1, only a very weak spectral shift was observed, indicating that either voriconazole does not bind to the enzyme at the concentrations employed (up to 100 μM) or the drug is not positioned in the P450 active site in the vicinity of the heme iron.

### Inhibition of Cholesterol 24S-Hydroxylase In Vitro

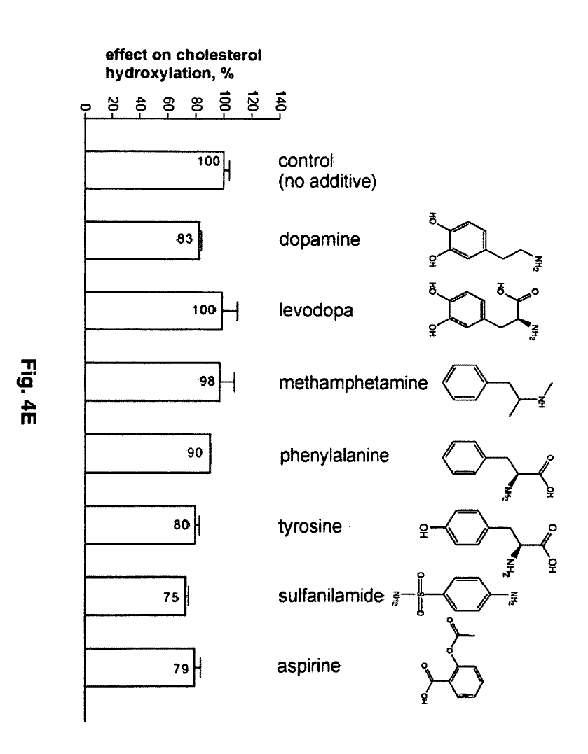

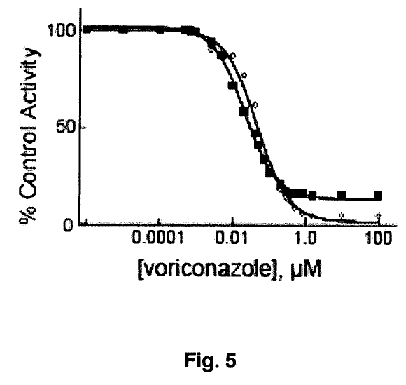

Of the three cholesterol-metabolizing P450s, CYP46A1 demonstrated the tightest binding of voriconazole. Therefore, whether voriconazole inhibits the enzyme activity in vitro was tested. CYP46A1 converts cholesterol to 24S-hydroxycholesterol and then can further metabolize 24S-hydroxycholesterol to 24, 24- and 24,27-dihydroxycholesterols. The IC50 for cholesterol 24-hydroxylation was 22 nM and that for 25- and 27-hydroxylations of 24S-hydroxycholesterol was 47 nM (FIG. 5). Since cholesterol concentration in the inhibition assay was equal to the Km, the Ki for cholesterol 24-hydroxylation was estimated to be 11 nM.

### Inhibition of Cholesterol 24S-Hydroxylase In Vivo

Mice were exposed to different doses of voriconazole, sacrificed experimental and control animals six hours after the injection and measured the levels of 24S-hydroxycholesterol in the brain. The treatment appeared to reduce the levels of 24S-hydroxycholesterol. The levels were slightly lower after the injection with 60 mg/kg body weight than after injection with 75 mg/kg body weight. In a subsequent experiment 5 mice with voriconazole were treated, 60 mg/kg body weight, and the animals and their controls killed 2 hours later. The levels of 24S-hydroxycholesterol were reduced by 20% but this effect was not statistically significant (p>0.05, Student's t-test). There was no significant effect on levels of lathosterol or total cholesterol in the brain.

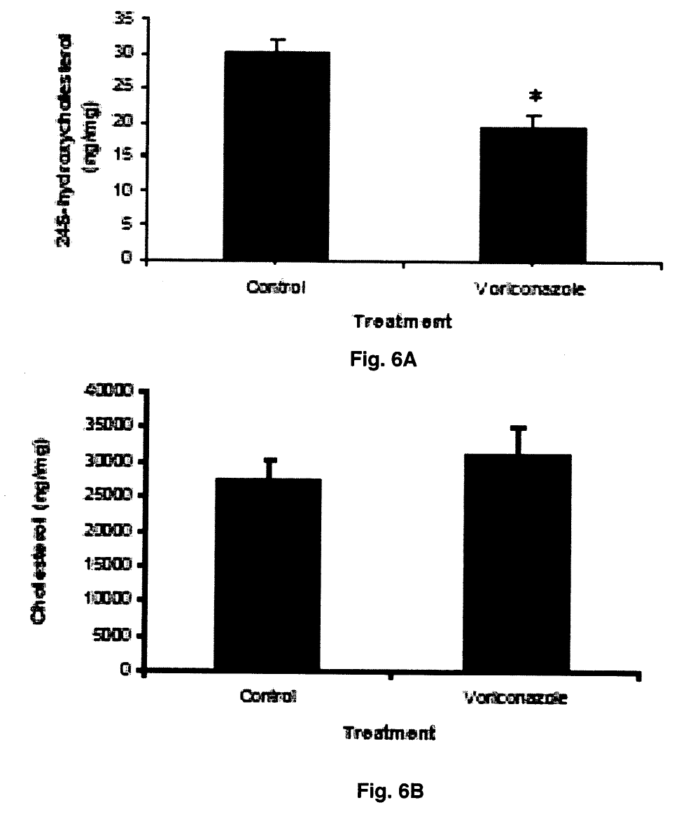

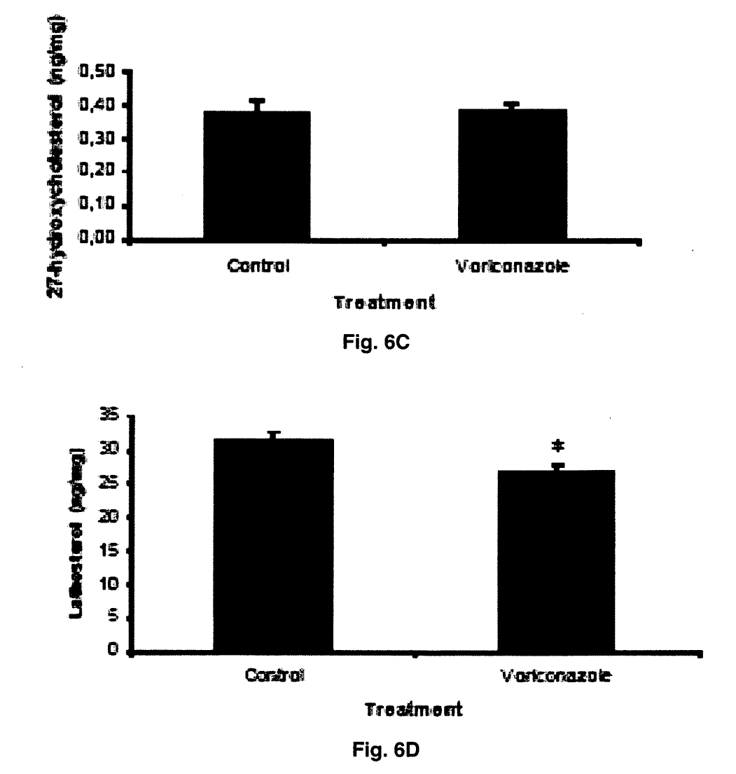

Mice were treated with voriconazole, 60 mg/kg body weight, once a day for 5 days, and a significant reduction (by 37%) was observed in the levels of 24S-hydroxycholesterol (p=0.03) (FIG. 6A). There was no effect on the levels of cholesterol (FIG. 6B) or 27-hydroxycholesterol (FIG. 6C) (p>0.05). However, the level of lathosterol in the brain was significantly reduced (FIG. 6D). Lathosterol serves as a marker for cholesterol biosynthesis. Therefore, reduced levels of this sterol in voriconazole-treated mice indicate a reduction in cholesterol synthesis (p<0.05).

Since most of the 24S-hydroxycholesterol present in the circulation originates from the brain, a reduction of brain synthesis should lead to a reduction of plasma 24S-hydroxycholesterol, unless voriconazole has an effect on the metabolism in the liver. A pool of plasma from the voriconazole-treated mice had a concentration of 24S-hydroxycholesterol of 13.2 ng/mL. A pool of plasma from the control mice had a concentration of 24S-hydroxycholesterol of 18.8 ng/mL. Thus, voriconazole appeared to suppress the plasma levels by about 30%. It should be pointed out that the plasma level of 27-hydroxycholesterol was not significantly affected by the voriconazole treatment. This oxysterol was at 46 ng/mL and 52 ng/mL in the voriconazole-treated mice and in the controls, respectively.

### Effects of Voriconazole on the Levels of Cholesterol Precursors Upstream of Lathosterol

The reduced levels of lathosterol in the voriconazole-treated mice suggest a reduced cholesterol synthesis. Part of this reduction could be due to the inhibition of the cytochrome P450 enzyme CYP51, which is responsible for demethylation of lanosterol, an upstream precursor of lathosterol. Brain levels of lanosterol increased from 6.1+2.0 ng/mg tissue in the controls to 27+10 ng/mg tissue in the voriconazole-treated mice (p<0.001). The levels of dehydrolanosterol were 0.14+0.05 ng/mg tissue and 1.9+0.7 ng/mg tissue, respectively. The levels of a lanosterol precursor squalene were 2.2+0.8 ng/mg tissue in the controls and 1.7+0.6 ng/mg tissue in the voriconazole-treated mice (p=0.05). The latter is consistent with a reduction of cholesterol synthesis also at a step prior to lanosterol demethylation.

Effects of Voriconazole on mRNA Levels of Genes Involved in Cholesterol Homeostasis

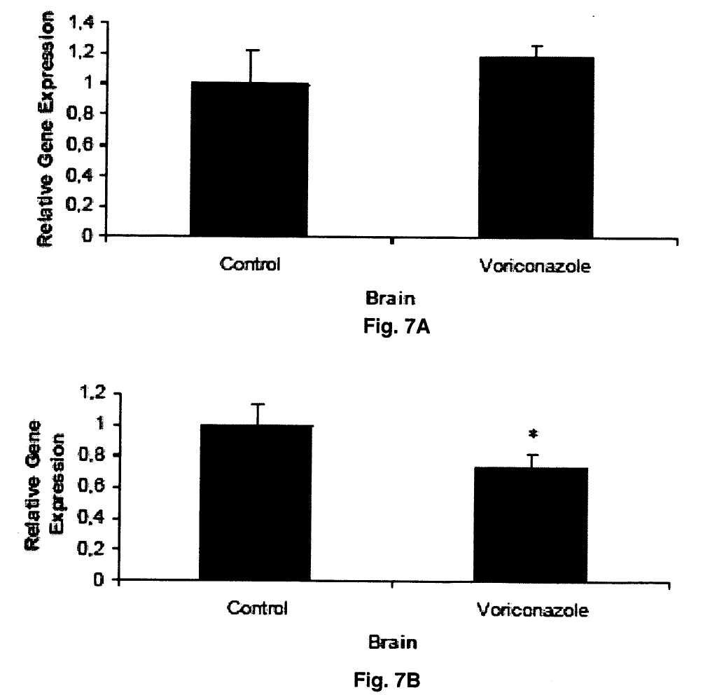

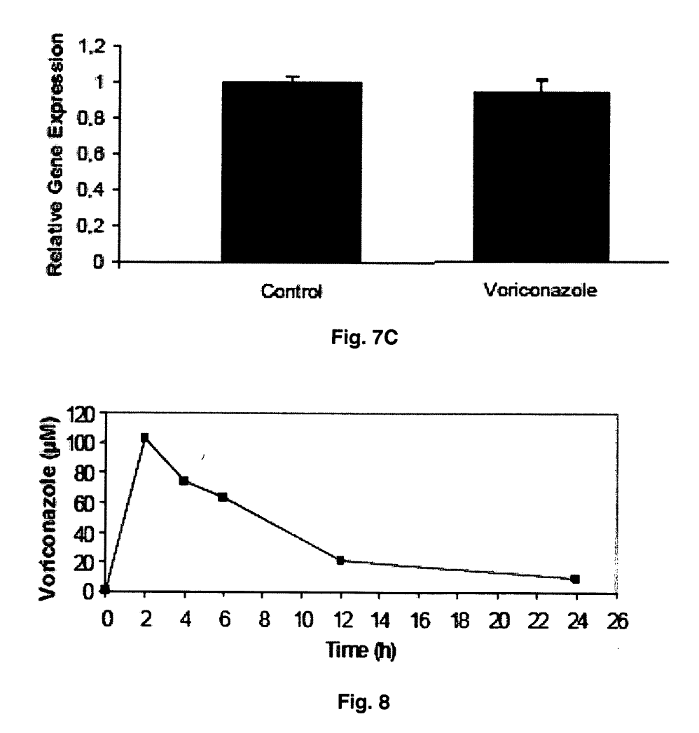

Voriconazole had no significant effect on expression of CYP46A1 mRNA (FIG. 7A) or HMG CoA synthase (FIG. 7C). However, there was a significant suppressive effect on the HMG CoA reductase mRNA levels (FIG. 7B). The latter is consistent with the inhibitory effect of voriconazole on the brain levels of squalene and lathosterol.

### Brain Levels of Voriconazole

The level of voriconazole in the brain of the mice after 5 daily intraperitoneal injections (60 mg/kg body weight) was 43+8 μg/g wet weight corresponding to 123 μM. Kinetics of brain levels of voriconazole after a single injection (60 mg/kg) is shown in FIG. 8. In the present study four antifungal azoles that are used systemically and three cholesterol-metabolizing P450s that are known to be expressed in the brain were tested. Voriconazole binds with high affinity to CYP46A1 in vitro and efficiently inhibits CYP46A1-catalyzed cholesterol hydroxylations in the reconstituted system. Then, in accordance with this finding and the fact that voriconazole can cross the blood-brain barrier, a statistically significant decrease in 24S-hydroxycholesterol levels in the brain in mice injected intraperitoneally with voriconazole for 5 days was detected. Also observed was a reduction in the levels of 24S-hydroxycholesterol in the circulation.

There are two different explanations for the reduced levels of 24S-hydroxycholesterol in the brain of the treated animals. The first explanation is a direct inhibition of the enzyme by voriconazole. The very high levels of voriconazole measured in the brain are in accord with this. The second possibility is related to the fact that voriconazole is an inhibitor of cholesterol synthesis. It has been shown that voriconazole exerts its antifungal effect through inhibition of cytochrome P450 14-alpha sterol demethylase, CYP51, which is responsible for the demethylation of lanosterol in the ergosterol biosynthesis pathway. Therefore, a reduced synthesis of the brain cholesterol may lead to a reduced substrate availability for CYP46A1 and reduced production of 24S-hydroxycholesterol.

The reduced levels of cholesterol precursors squalene and lathosterol as well as HMG CoA reductase mRNA indicate that brain cholesterol synthesis was reduced in the voriconazole-treated mice. The fact that the treatment had no significant effect on the size of the pool of cholesterol in the brain, does not favour the hypothesis that reduced substrate availability is of importance for the reduced production of 24S-hydroxycholesterol. A primary effect of voriconazole on CYP46A1 can be expected to lead to a reduced metabolism of brain cholesterol and thus a reduced need for de novo synthesis. If the reduced rate of lanosterol demethylation had been the most important effect, a compensatory increase of HMG CoA reductase mRNA would have been expected.

The expression of the HMG CoA reductase gene is regulated by SREBP-2, and we had expected a significant effect on expression of another SREBP-2 regulated gene, HMG CoA synthase. Our failure to demonstrate this may be related to the interindividual variations which may be too great to detect a relatively small difference. Addition of 24S-hydroxycholesterol to cultures of primary neuronal cells from embryonic rat pups was also shown to significantly decrease the protein level of HMG CoA synthase. Levels of cholesterol are low in such embryonic cells and the cultures were carried out in cholesterol-deficient medium. Therefore, the rate of cholesterol synthesis should be high. These conditions are markedly different from those of adult neuronal cells in vivo, and the levels of transcriptional factors may be very different in embryonic cells.

The following references are cited herein:
1. Lund et al., 1999, Proc Natl Acad Sci USA, 96:7238-7243.
2. Bjorkhem et al., 1998, J. Lipid Res, 39:1594-1600.
3. Bjorkhem et al., 2001, J Biol Chem, 276:37004-37010.
4. Kotti et al., 2006, Proc Natl Acad Sci USA, 103:3869-3874.
5. Kolsch et al., 2002, Mol Psychiatry, 7:899-902.
6. Papassotiropoulos et al., 2003, Arch Neurol, 60:29-35.
7. Borroni et al., 2004, Neurobiol Aging, 25:747-751.
8. Combarros et al., 2004, Dement Geriatr Cogn Disord, 18, 257-260.
9. Johansson et al., 2004, Hum Genet, 114:581-587.
10. Wang et al., 2004, Neurosci Lett, 369:104-107.
11. Papassotiropoulos et al., 2005, J Clin Psychiatry, 66:940-947.
12. Golanska et al., 2005, Neurosci Lett, 383:105-108.
13. Fernandez Del Pozo et al., 2006, Dement Geriatr Cogn Disord, 21:81-87.
14. Helisalmi et al., 2006, Neurosurg Psychiatry, 77:421-422.
15. Desai et al., 2002, Neurosci Lett, 328:9-12.
16. Chalmers et al., 2004, NeuroReport, 15:95-98.
17. Ingelsson et al., 2004, Neurosci Lett, 367:228-231.
18. Kabbara et al., 2004, Neurosci Lett, 363:139-143.
19. Juhasz et al., 2005, Neurochem Res, 30:943-948.
20. Shibata et al., 2006, Neurosci Lett, 391:142-146.
21. Bogdanovic et al., 2001, Neurosci Lett, 314:45-48.
22. Brown et al., 2004, J Biol Chem, 279:34674-34681.
23. Mast et al., 2003, Biochemistry, 42:14284-14292.
24. Ohyama et al., 2006, J Biol Chem, 281:3810-3820.
25. Janowski et al., 1999, Proc Natl Acad Sci USA, 96:266-271.
26. Scott et al., 2004, J Biol Chem, 279:27294-27301.
27. Brunger et al., 1998, Acta Crystallogr D Biol Crystallogr, 54:905-921.
28. Lovell et al., 2003, Proteins, 50:437-450.
29. Mast et al., 2004, Arch Biochem Biophys 428:99-108.
30. Copeland, R. A., 2000, in Protein-Lignad Binding Equilibria, (Copeland R. A., ed. Enzymes), 2^{nd} ed. New York: A John Wiley & Sons, Inc, pg. 76-108.
31. Chen et al., 2007, Proteins, 67:593-605.
32. Kellog, G. and Chen, D. L., 2003, J Pharmacol Exp Ther, 307:878-887.
33. Cozzini et al., 2002, J. Med. Chem., 45:2469-2483.
34. Formabaio et al. 2003, J. Med. Chem., 46:4487-4500.
35. Formabaio et al. 2004, J. Med. Chem., 47:4507-4516.
36. Morris et al., 1998, J. Comput. Chem., 19:1639-1662.
37. Morris et al. 1996, J. Comput. Aided Mol. Des., 10:293-304.
38. Kleywert and Jones, 1994, Acta Crystallogr D Biol Crystallogr 50:178-185.
39. Murray M. and Wilkinson C. F., 1984, Chem Biol Interact 50:267-275.
40. Mallinger et al., 1996, Clin Pharmacol Ther 40:444-450.
41. Hutzler et al., 2001, Drug Metab Dispos 29:1029-1034.
42. Wester et al., 2004, J Biol Chem 279:35630-35637.
43. Locuson et al. 2007 J Med Chem 50:1158-1165.

Any patents or publications mentioned in this specification are indicative of the level of those skilled in the art to which the invention pertains. Further, these patents and publications are incorporated by reference herein to the same extent as if each individual publication was specifically and individually incorporated by reference.

One skilled in the art would appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

What is claimed is:

1. A method for controlling an activity of a cytochrome P450 46A1 (CYP46A1) enzyme, comprising:
contacting the CYP46A1 enzyme with a compound that binds within the CYP46A1 active site such that cholesterol hydroxylation is effectively inhibited or stimulated thereby controlling the CYP46A1 activity.

2. The method of claim 1, wherein the compound is a CYP46A1 enzyme inhibitor binding within a substrate-binding site of the enzyme active site.

3. The method of claim 2, wherein the inhibitor is a compound comprising selected from the group consisting of a sulfate moiety, a sulfonamide moiety, an azole moiety, a histamine receptor antagonist, a monoamine oxidase inhibitor, or combinations thereof.

4. The method of claim 3, wherein the sulfate-containing compound is selected from the group consisting of cholesterol sulfate, pregnenolone sulfate, estradiol sulfate, testosterone sulfate and DHEA sulfate.

5. The method of claim 3, wherein the sulfonamide-containing compound is famotidine or sulfanilamide.

6. The method of claim 3, wherein the azole-containing compound is an antifungal compound.

7. The method of claim 6, wherein the antifungal compound is voriconazole or clotrimazole.

8. The method of claim 3, wherein the histamine receptor antagonist is famotidine, nizatidine, cimetidine, ranitidine, thioperamide, or clobenpropit.

9. The method of claim 3, wherein the monoamine oxidase inhibitor is selegiline or tranylcypromide.

10. The method of claim 2, wherein the inhibitor is retinol or aspirin.

11. The method of claim 1, wherein the compound is a CYP46A1 enzyme stimulator binding within a subpocket of the enzyme active site without interfering with cholesterol binding.

12. The method of claim 11, wherein the subpocket is formed by at least residues L112, F121, V126, L219, I222, I301, A302, A474, and T475.

13. The method of claim 11, wherein the stimulator is a non-steroidal compound.

14. The method of claim 13, wherein the non-steroidal compound is acetaminophen and phenacetin.

15. A method for designing a potential regulator compound of a post-translational CYP46A1 activity, comprising:
identifying a test compound that interacts within the active site of CYP46A1, said identification based at least in part on the crystal structure of CYP46A1.

16. The method of claim 15, further comprising:
screening the test compounds for regulation of a post translational activity of CYP46A1 enzyme.

17. The method of claim 16, comprising:
selecting a designed test compound that interacts with the active site of CYP46A1 enzyme;
contacting the CYP46A1 enzyme with the test compound and cholesterol or with cholesterol alone;
measuring the level of cholesterol hydroxylation in the presence and in the absence of the test compound; and
comparing the level of cholesterol hydroxylation in the presence of the test compound with the level of cholesterol hydroxylation in the absence of the test compound, wherein a decrease in cholesterol hydroxylation in the presence of the test compound is indicative that the test compound is an inhibitor of CYP46A1 activity or wherein an increase in cholesterol hydroxylation in the presence of the test compound is indicative that the test compound is a stimulator of CYP46A1 activity, said inhibitor or stimulator compound thereby regulating CYP46A1 activity post-translationally.

18. The method of claim 17, wherein the CYP46A1 inhibitor or stimulator compound crosses the blood brain barrier or the blood retina barrier.

19. The method of claim 15, wherein the crystal structure is 2Q9G and the test compound is an inhibitor of cholesterol hydroxylation by CYP46A1, said inhibitor binding within a substrate binding site in 2Q9G.

20. The method of claim 15, wherein the crystal structure is 2Q9F and the test compound is a stimulator of cholesterol hydroxylation by CYP46A1, said stimulator binding within a subpocket of the CYP46A1 active site in 2Q9G.

21. The method of claim 20, wherein the subpocket is formed by at least residues Ile301, Val215, Ile219, Ile222, Als 474, Leu112, Leu120, and Phe121.

22. A regulator compound of CYP46A1 post-translational activity identified by the method of claim 15.

23. The regulator compound of claim 22, wherein the regulator compound is an inhibitor selected from the group consisting of a sulfate moiety, a sulfonamide moiety, an azole moiety, a histamine receptor antagonist and a monoamine oxidase inhibitor.

24. The regulator compound of claim 22, wherein the regulator is a non-steroidal stimulator compound having an aromatic or aryl structure.

25. A method for treating a pathoneurological condition associated with increased cholesterol levels in the brain or retina of a subject, comprising:
administering to the subject a pharmacologically effective amount of the stimulator compound of claim 22, said stimulator compound increasing hydroxylation of cholesterol by the CYP46A1 enzyme in the brain or retina thereby decreasing cholesterol levels therein to treat the pathoneurological condition in the subject.

26. The method of claim 25, wherein increased cholesterol levels result from the binding of another drug to the substrate binding site of the CYP46A1 enzyme.

27. The method of claim 25, wherein administering the stimulator compound delays or prevents onset of the pathoneurological condition in the subject.

28. The method of claim 25, wherein the pathoneurological condition is Alzheimer's disease, dementia, deficiency in spatial, associative and motor learning, age-related macular degeneration.
